(19) United States
(12) Patent Application Publication
(10) Pub. No.: US 2025/0248619 A1
(43) Pub. Date: Aug. 7, 2025

(54) MEASURING APPARATUS AND METHOD FOR EVALUATING FOOT HEALTH

(71) Applicant: The Board of Regents of the University of Oklahoma, Norman, OK (US)

(72) Inventors: Hazem H. Refai, Tulsa, OK (US); Elizabeth S. Hile, Edmond, OK (US); Josiah R. Rippetoe, Oklahoma City, OK (US); Abby Cha, Norman, OK (US); Mohamad Yazan Sedoun, Tulsa, OK (US)

(73) Assignee: The Board of Regents of the University of Oklahoma, Norman, OK (US)

(21) Appl. No.: 19/038,420

(22) Filed: Jan. 27, 2025

Related U.S. Application Data
(60) Provisional application No. 63/729,222, filed on Dec. 6, 2024.
(60) Provisional application No. 63/676,817, filed on Jul. 29, 2024.
(60) Provisional application No. 63/627,850, filed on Feb. 1, 2024.

(51) Int. Cl. A61B 5/103; A61B 5/00; A61B 5/1455

(52) U.S. Cl. CPC A61B 5/1036; A61B 5/14551; A61B 5/6829

(57) ABSTRACT

A foot evaluation device is configured to evaluate metrics of foot health accurately and precisely. The foot evaluation device includes a platform configured to accommodate a user's foot, and a toe sensor assembly that is removably attachable to the platform and includes a toe force sensor and a toe cap connected to the toe force sensor. The toe force sensor is configured to measure forces applied to the toe force sensor by a toe of the user's foot.

### RELATED APPLICATIONS

This application claims the benefit of U.S. Provisional Patent Application No. 63/729,222 filed Dec. 6, 2024, and entitled “Measuring Apparatus and Method for Evaluating Foot Health”, U.S. Provisional Patent Application No. 63/676,817 filed Jul. 29, 2024, and entitled “Toe Flexion and Extension Measuring Apparatus and Method”, and U.S. Provisional Patent Application No. 63/627,850 filed Feb. 1, 2024, and entitled “Measuring Apparatus and Method for Evaluating Foot Health”, the disclosures of which are herein incorporated by reference.

### BACKGROUND

The strength and overall condition of one's foot serves as an important indicator of health. For example, toe strength and range of motion may be used to diagnose or evaluate a number of conditions, including cancer, diabetes, vascular disease, and distal neuropathies or myopathies of any etiology. In the past, physical therapists, oncologists, neurologists and other healthcare providers have used manual examination techniques to evaluate foot health, and these techniques are inherently subjective and prone to discrepancies between clinicians. Although some measurement devices are available to capture more consistent results, these devices are limited in both accuracy and convenience. Further, these devices have a limited ability to assess overall foot health.

For example, current measurement devices are unable to detect whether a patient has performed a pure toe flexion or toe extension movement without the assistance of other parts of the foot. To achieve accurate toe strength measurements, the toe should be flexed and extended without using other parts of the foot. If inaccurate measurements are obtained because a patient used his whole foot during these toe strength measurements, a loss in toe strength or range of motion may go undetected. The inability to monitor the overall foot while performing a toc strength measurement makes it difficult to determine when an accurate toe strength measurement has been achieved. Current toe strength measurement devices also fail to provide a bigger picture of foot muscle strength, foot shape, and progression toward conditions such as flat foot.

Current measurement devices are also sensitive and often inaccessible to untrained users. The ability to monitor and report whether toe measurements are being reliably performed would permit partial or total automation of the measurement acquisition process, such that an unskilled user could evaluate foot health without the assistance of an on-site clinician.

There is, therefore, a need for an improved mechanism for evaluating aspects of overall foot health including, but not limited to, toe strength and range of motion. The present disclosure is directed to addressing these and other deficiencies in the prior art.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function.

FIG. 1 is a top perspective view of a foot evaluation device constructed in accordance with an exemplary embodiment.

FIG. 2 is a side view of the foot evaluation device of FIG. 1.

FIG. 3 is a top perspective view of an embodiment of the foot evaluation device having a heel force sensor and a metatarsal force sensor.

FIG. 4 is a top perspective view of an embodiment of the foot evaluation device having a heel force sensor.

FIG. 5 is a top perspective view of an embodiment of the foot evaluation device having a metatarsal force sensor.

FIG. 6 is a top perspective view of an embodiment of the foot evaluation device having a foot placement adjustment system.

FIG. 7 is a bottom perspective view of the foot evaluation device of FIG. 6.

FIG. 8 is a top perspective view of an embodiment of a latch assembly of the foot evaluation device.

FIG. 9 is a front perspective view of the latch assembly of FIG. 8 in connection with an example toe sensor assembly, where the latch assembly is in an unlocked position.

FIG. 10 is a front perspective view of the latch assembly of FIG. 8 in connection with the example toe sensor assembly of FIG. 9, where the latch assembly is in a locked position.

FIG. 11A is a side perspective view of an example toe sensor assembly of the foot evaluation device.

FIG. 11B is a side view of the toe sensor assembly of FIG. 11A attached to a platform of the foot evaluation device.

FIGS. 12A through 12C depict side views of the toe sensor assembly of FIG. 11A in use with platforms and toe force sensors of different sizes.

FIG. 13 is a top perspective view of another example toe sensor assembly of the foot evaluation device.

FIG. 14 is a top perspective view of another example toe sensor assembly of the foot evaluation device.

FIG. 15A is a top perspective view of yet another example toe sensor assembly of the foot evaluation device.

FIG. 15B is a top perspective view of yet another example toe sensor assembly of the foot evaluation device.

FIG. 16 is a top perspective view of an embodiment of the foot evaluation device configured with a pressure sensor array.

FIG. 17 is a top perspective view of an embodiment of the foot evaluation device configured with a pressure sensor array, a heel force sensor, and a metatarsal force sensor.

FIG. 18 is a top perspective view of an embodiment of the foot evaluation device configured with an optical sensor array.

FIG. 19 is a top perspective view of an embodiment for a pedal attachment of the foot evaluation device.

FIG. 20 is a top perspective view of an embodiment of the foot evaluation device configured with a pedal attachment.

### DETAILED DESCRIPTION

The present disclosure is directed, in non-limiting embodiments, to a foot evaluation device configured to evaluate metrics of foot health accurately and precisely. In one aspect, the foot evaluation device includes a platform configured to accommodate a user's foot and a toe sensor assembly that is removably attachable to the platform. The toe sensor assembly includes a toe force sensor and a toe cap connected to the toe force sensor. The toe force sensor is configured to measure forces applied to the toe force sensor by a toe of the user's foot.

In another aspect, a method for evaluating a user's foot with a foot evaluation device is disclosed. The method involves the steps of attaching a toe sensor assembly to a platform of the foot evaluation device, positioning a toe of the user's foot in contact with a toe force sensor of the toe sensor assembly, and instructing the user to execute a measurement protocol that includes toe flexion exercises, toe extension exercises, or a combination of toe flexion and toe extension exercises. The method also includes the steps of recording toe-related measurements resulting from the application of force by the user's toe on the toe force sensor during the measurement protocol and detaching the toe sensor assembly from the platform.

In yet another aspect, the foot evaluation device includes a platform configured to accommodate a user's foot; a foot placement adjustment system configured to minimize movement of the user's foot on the platform; at least one toe sensor assembly that is removably attachable to the platform; a heel force sensor positioned along the platform to contact the heel of the user's foot when the toe is within the toe sensor assembly; and a metatarsal force sensor positioned along the platform to contact the metatarsal region of the user's foot when the toe is within the toe sensor assembly. The heel force sensor is configured to measure and produce heel-related output signals representative of forces applied by the user's foot to the heel force sensor during toe flexion and toe extension, and the metatarsal force sensor is configured to measure and produce metatarsal-related output signals representative of forces applied by the user's foot to the metatarsal force sensor during toe flexion and toe extension. The foot evaluation device also includes a pressure sensor array that has a plurality of pressure sensors that contact the user's foot at different points on the platform and an optical sensor array that has a plurality of light emitters and a plurality of photodiodes that contact the user's foot on the platform. The foot evaluation device also includes a control module that is configured to estimate the accuracy of measurements taken by the toe sensor assembly using the heel-related output signals and the metatarsal-related output signals and a pedal attachment that interfaces with the control module. The pedal attachment includes a foot pedal; a potentiometer connected to the foot pedal, where the potentiometer measures an applied force by the user's foot to the foot pedal; a linear actuator connected to the foot pedal; and a processor in communication with the potentiometer and the linear actuator.

Before describing various embodiments of the present disclosure in more detail by way of exemplary description, examples, and results, it is to be understood as noted above that the present disclosure is not limited in application to the details of methods and apparatus as set forth in the following description. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the methods and apparatus of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges (e.g., in units of length such as micrometers or millimeters) include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure. More particularly, a range of 10-12 units includes, for example, 10, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, and 12.0, and all values or ranges of values of the units, and fractions of the values of the units and integers within said range, and ranges which combine the values of the boundaries of different ranges within the series, e.g., 10.1 to 11.5.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is to be understood that the terms “including”, “comprising”, “consisting” and grammatical variants thereof do not preclude the addition of one or more components, features, steps, or integers or groups thereof and that the terms are to be construed as specifying components, features, steps or integers.

It is to be understood that where the specification states that a component, feature, structure, or characteristic “may”, “might”, “can” or “could” be included, that particular component, feature, structure, or characteristic is not required to be included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error. Further, in this detailed description, each numerical value (e.g., temperature or time) should be read once as modified by the term “about” (unless already expressly so modified), and then read again as not so modified unless otherwise indicated in context. As noted above, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range. Unless otherwise stated, the term “about” or “approximately”, where used herein when referring to a measurable value such as an amount, length, thickness, a temporal duration, and the like, is meant to encompass, for example, variations of +20% or +10%, or +5%, or +1%, or +0.1% from the specified value, as such variations are appropriate to perform the disclosed methods and as understood by persons having ordinary skill in the art.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement (e.g., length).

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein any reference to “we” as a pronoun may include laboratory personnel or other contributors who assisted in the laboratory procedures and data collection and is not intended to represent an inventorship role by said laboratory personnel or other contributors in any subject matter disclosed herein.

Where applicable, although state diagrams, flow diagrams or both may be used to describe embodiments, the present disclosure is not limited to those diagrams or to the corresponding descriptions. For example, flow need not move through each illustrated box or state, or in exactly the same order as illustrated and described. Methods of the present disclosure may be implemented by performing or completing manually, automatically, or a combination thereof, selected steps or tasks. The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility). Still further, additional aspects of the various embodiments of the instant disclosure may be found in one or more appendices attached hereto and/or filed herewith, the disclosures of which are incorporated herein by reference as if fully set out at this point.

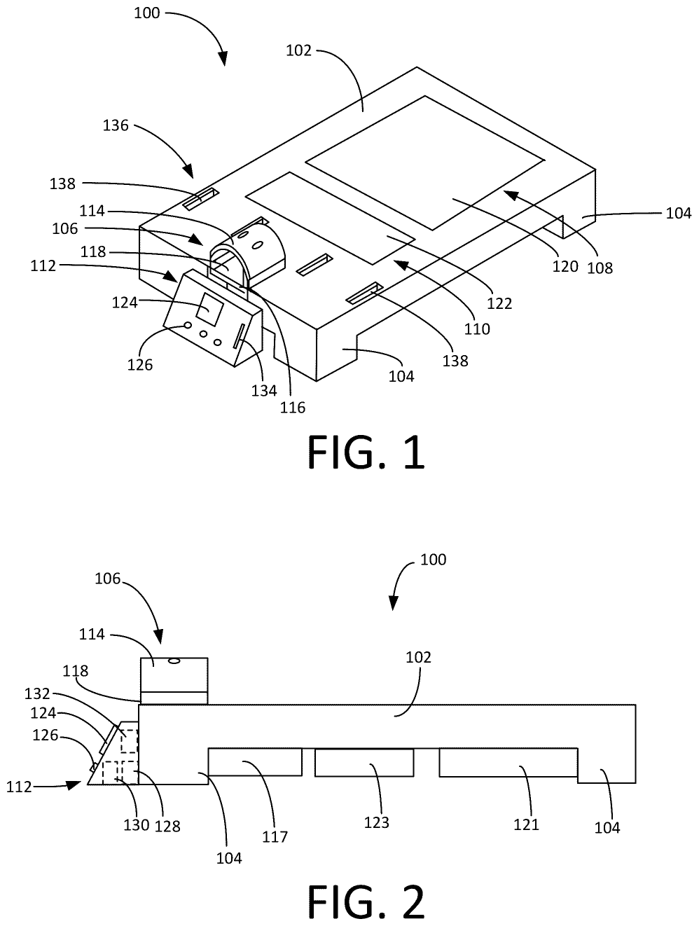

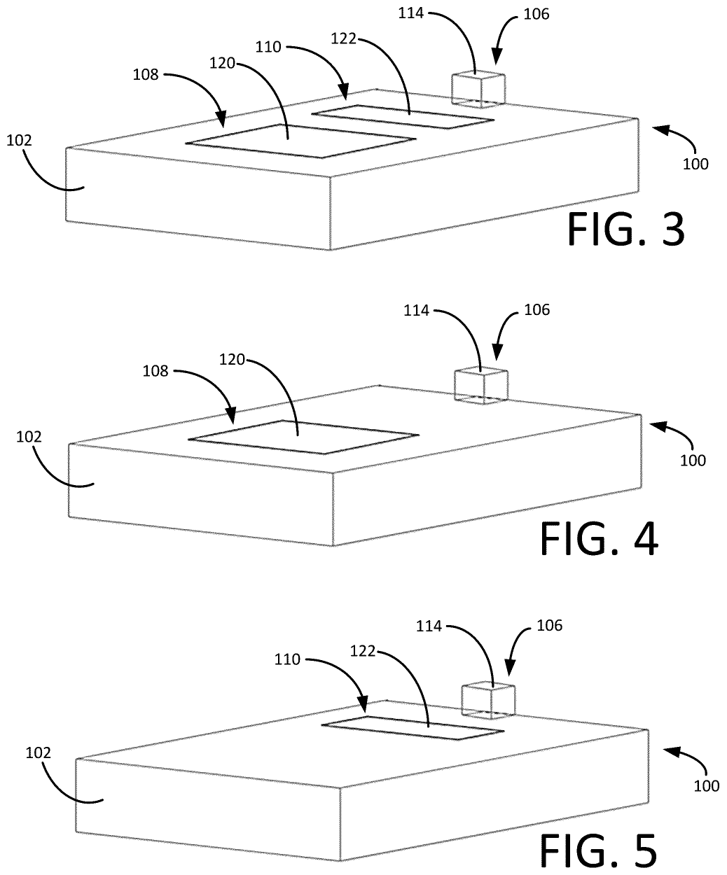

Referring now to FIGS. 1 and 2, shown therein are top perspective and side views, respectively, of a foot evaluation device 100 constructed in accordance with a first embodiment. The foot evaluation device 100 has a platform 102 with a plurality of feet 104 that support the platform 102 on a floor or other surface. As shown in FIGS. 3 through 5, in other non-limiting embodiments the platform 102 is not supported by feet 104 and is, instead, configured for direct contact with the floor or other surface. The foot evaluation device 100 of FIGS. 1 and 2 includes a toe sensor assembly 106, a heel force sensor 108, a metatarsal force sensor 110, and a control module 112. The platform 102 is sized and configured to accommodate a user's left or right foot such that the first toe (sometimes referred to as the “big toe,” “great toe” or “hallux”) on either foot can be easily placed within the toe sensor assembly 106 that is centrally located proximate to an edge of the platform 102. The platform 102 may be fabricated in various sizes to accommodate larger or smaller feet or may be sized to accommodate a specific shoe size. When the user's first toe is placed in a measurement position within the toe sensor assembly 106, the heel of the user's foot will be positioned over the heel force sensor 108, and the metatarsal region of the same foot will be positioned over the metatarsal force sensor 110. It will be appreciated that the force sensors 108, 110 may be sized to extend over a relatively larger or smaller surface area of the platform than is depicted in FIG. 1.

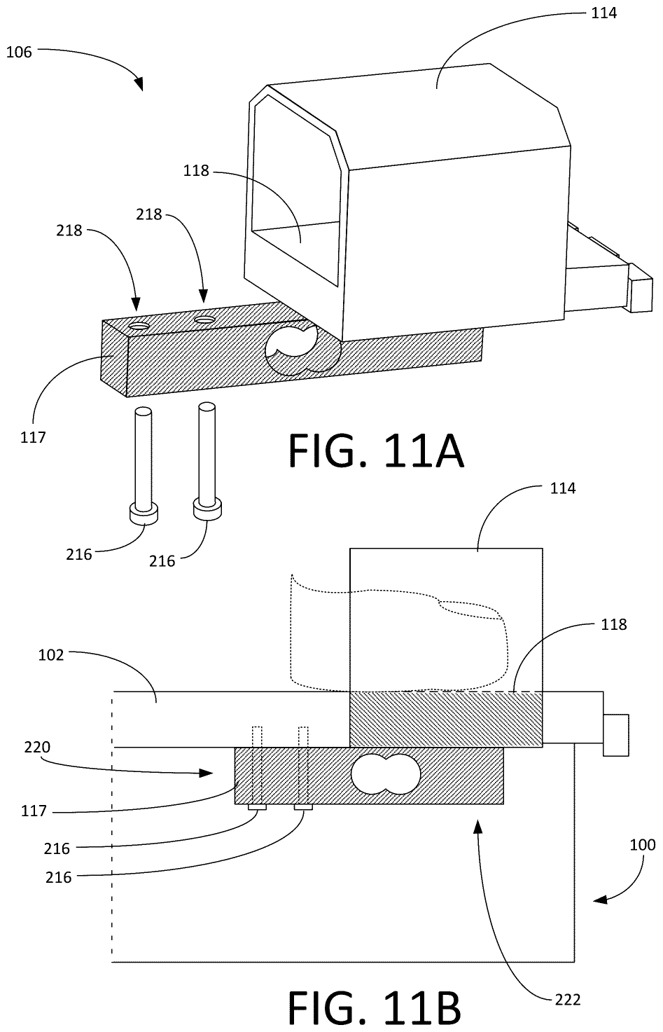

The toe sensor assembly 106 is retained on, within, or against the platform 102. In exemplary embodiments, as depicted in FIGS. 1-2 and 11-12, the toe sensor assembly 106 includes a toe cap 114 connected to a toe force sensor 116. The toe force sensor 116 uses an electronic load cell or scale 117 to obtain force measurements and has a toe sensor surface 118 to which the toe cap 114 is connected. The toe sensor surface 118 of the toe force sensor 116 may be substantially flush with the top surface of the platform 102. The toe cap 114 of the toe sensor assembly 106 can be constructed from a rigid or semi-rigid material, or from a flexible fabric or synthetic material that can be tightened around the user's toe. In exemplary embodiments, the toe cap 114 is configured to be easily exchanged so that various sizes and types of toe caps 114 can be quickly connected to the toe force sensor 116 to accommodate variations in user anatomy. The toe cap 114 is preferably adjusted such that there is little or no space between the top of the user's first toe and the inside of the toe cap 114 when the user's toe is planted in a neutral position on the toe force sensor 116. This ensures that the entire range of movement of the user's toe is detectable by the toe sensor assembly 106 when the toe is in a measurement position.

The load cell 117 of the toe force sensor 116 is configured to measure the magnitude and direction of a force applied to the toe sensor surface 118 and output a responsive toe-related output signal. If force is applied against the toe sensor surface 118 during toc flexion, the toe force sensor 116 measures and reports the magnitude of the force in a positive direction. If the toc cap 114 is pulled away from the toe force sensor 116 during toe extension, the toe force sensor 116 measures and reports the magnitude of force in a negative direction. In both cases, the toe force sensor 116 measures the magnitude and direction of force in real time with a sampling rate that permits multiple measurements to be taken over a short period of time (e.g., 100 measurements/second). In some embodiments, the sampling rate, the sampling time duration, and corresponding data file size can be adjusted with the control module 112. Although the toe sensor assembly 106 is well-suited for measuring forces exerted by the first toe, it will be appreciated that the toe sensor assembly 106 can also be configured in combination with the other components of the foot evaluation device 100 to measure forces applied by one or more other toes.

The heel force sensor 108, which can be used to confirm whether measurements from the toe sensor assembly 106 accurately characterize toe strength, may be used alone (FIG. 4) or in combination with the metatarsal force sensor 110 (FIG. 3). The heel force sensor 108 may be integrated with the platform 102 or configured as a removable component of the foot evaluation device 100. In exemplary embodiments, as depicted in FIGS. 2 and 7, the heel force sensor 108 has a heel sensor surface 120 that is substantially flush with the top surface of the platform 102 and uses an electronic load cell or scale 121 to obtain force measurements. The heel sensor surface 120 is positioned to contact the heel of the user's foot when the user's toe is in the measurement position within the toe sensor assembly 106.

The incorporation of the heel force sensor 108 allows for detection of the forces exerted by the user's heel during toe flexion and during toe extension. During toc flexion and toc extension, the heel force sensor 108 is configured to measure and report both the magnitude and direction of the force applied to the heel sensor surface 120. The heel force sensor 108 performs measurements with a high sampling rate (e.g., 100 measurements/second) and produces a heel-related output signal in response to detected force on the heel sensor surface 120. In some embodiments, the control module 112 of the foot evaluation device 100 can be used to adjust sampling rate of the heel force sensor 108, sampling time duration, and corresponding data file size. A series of heel-related output signals is representative of the magnitude of the forces applied over time.

In exemplary embodiments, the metatarsal force sensor 110 helps to demonstrate whether a measurement from the toe sensor assembly 106 accurately characterizes toe strength. As depicted in FIGS. 3 and 5, the metatarsal force sensor 110 may be used either alone or in combination with the heel force sensor 108. The metatarsal force sensor 110 may either be integrated with the platform 102 or a removable component. The metatarsal force sensor 110 of certain non-limiting embodiments has a metatarsal sensor surface 122, uses an electronic load cell or scale 123 to obtain force measurements, and is positioned such that the metatarsal sensor surface 122 contacts the user's metatarsal (the region between the user's heel and toe) when the user's toe is in the measurement position within the toe sensor assembly 106. The metatarsal force sensor 110 detects forces exerted by the user's metatarsal on the metatarsal sensor surface 122. The metatarsal force sensor 110 is configured to measure both the magnitude and direction of the force applied to the metatarsal sensor surface 122 during toe flexion, during toe extension, or both. This information is reported as metatarsal-related output signals to the control module 112. The sampling rate of the metatarsal force sensor 110 permits multiple measurements to be taken in real time over a short period (e.g., 100 measurements/second). In some embodiments, the sampling rate, the sampling time duration, and corresponding data file size are adjustable through the control module 112. A series of metatarsal-related output signals is representative of the magnitude of the forces applied by the user's foot over time. Metatarsal-related output signals can also be used, either alone or in combination with heel-related output signals, to analyze and report a relationship with toe force parameters.

The toe-related output signals from the toe sensor assembly 106 are provided to the control module 112 (along with any heel-related and/or metatarsal-related output signals) to be recorded, processed, stored, and displayed. The control module 112 includes a display 124, control buttons 126, a battery 128, a processor 130, memory 132, and an output module 134. FIGS. 1 and 2 depict the control module 112 in direct attachment onboard the foot evaluation device 100. In another embodiment, all or a portion of the control module 112 is connected to the foot evaluation device 100 over a wired or wireless (e.g., Wi-Fi or Bluetooth) connection. In some embodiments, the control module 112 is a separate tablet, computer, or handheld device that communicates with the foot evaluation device 100 over a wired or wireless connection.

The heel force sensor 108 and the metatarsal force sensor 110 allow the clinician to monitor the accuracy of measurements made at the toe force sensor 116. Output signals from the heel force sensor 108 and the metatarsal force sensor 110 allow the clinician to discriminate between forces applied only by isolated movement of the user's toe and forces generated by the user's toc in combination with other foot movements. This allows the clinician to detect situations where the forces measured by the toe sensor assembly 106 are inaccurate because the measurement results from broader foot movements. For example, rather than flexing the toe, a user might push the entire foot forward while locking the toe to apply a force against the toe force sensor 116 that mimics the intended toe flexion measurement. When this occurs, the heel force sensor 108 and metatarsal force sensor 110, if present, will generate output signals that indicate changes in forces applied to the heel sensor surface 120 and metatarsal sensor surface 122. The processor 130 of the control module 112 processes the heel-related and metatarsal-related output signals, determines whether the detected changes in applied force relate to forward foot movement, and thereby estimates the accuracy of the measurement taken by the toe sensor assembly or indicates that the related toe measurement is inaccurate. As another example, the user might push the heel downward as the toe is being extended, thereby artificially increasing the distance of toe extension. In this case, the heel-related output signals indicate the increased downward force on the heel force sensor 108, and the control module 112 flags the toe extension measurement for a lack of accuracy. In some embodiments, the control module 112 can be configured to automatically correct, scale or otherwise adjust the output of the toe sensor assembly 106 to account for foot movements detected by the heel force sensor 108 and metatarsal force sensor 110.

If a user is unable to perform toe extension or toe flexion without applying force through the heel or metatarsal, the control module 112 can be configured to use measurements taken by the heel force sensor 108 and metatarsal force sensor 110 to confirm a baseline for at least one earlier measurement of the toe force sensor 116. The processor 130 can also analyze the output signals to report a relationship for toe measurements with heel and/or metatarsal force parameters. In subsequent measurements, the clinician can determine if forces applied to the toe sensor assembly 106 represent a clinical change, particularly if the measurements made by the heel force sensor 108 and metatarsal force sensor 110 remain substantially the same across the series of tests using the foot evaluation device 100. Measurements from the heel force sensor 108 and the metatarsal force sensor 110 may also be used to reveal that the user is using the heel or metatarsal to compensate for a toe weakness or to confirm a change of pattern indicative of a toc weakness. In this way, the heel force sensor 108 and metatarsal force sensor 110 provide contextual measurements that can be valuable in more accurately measuring isolated changes in forces applied by the user to the toe sensor assembly 106.

The display 124 is configured to display the operational status of the foot evaluation device 100, the real-time measurements from the toe force sensor 116, the estimated accuracy of the toe-related measurements, charge levels in the battery 128, and other messages produced by the processor 130. For example, the display 124 may provide real-time visualization of one or more sensor outputs to the user, which may encourage the user to maximize effort or allow for the accuracy of the measurement to be inspected. It will be appreciated that the display 124 is optional and not included in certain embodiments. In the embodiment of FIGS. 1 and 2, the display 124 is pictured onboard the foot evaluation device 100. In other embodiments, the display 124 is the screen of a separate tablet, computer, or handheld device that is connected by a wire or wirelessly to the foot evaluation device 100. The separate tablet, computer, or handheld device optionally runs a mobile application to allow remote communication with the control module 112 for configuration of the foot evaluation device 100, data display, and recording. The battery 128 is located inside the control module 112 or elsewhere within the foot evaluation device 100. In some embodiments, the foot evaluation device 100 is configured to be powered by standard grid or wall power. The control buttons 126 may include a power button, a tare button, and a record button for initiating a recording session. Navigation buttons may be provided to cycle through menus, settings, and data files stored within the memory 132. In some embodiments, the foot evaluation device 100 is configured to automatically power on when an initial force is measured by the toe force sensor 116. The foot evaluation device 100 can be configured to automatically power down after the passage of a predetermined period without the detection of a load by the toe force sensor 116.

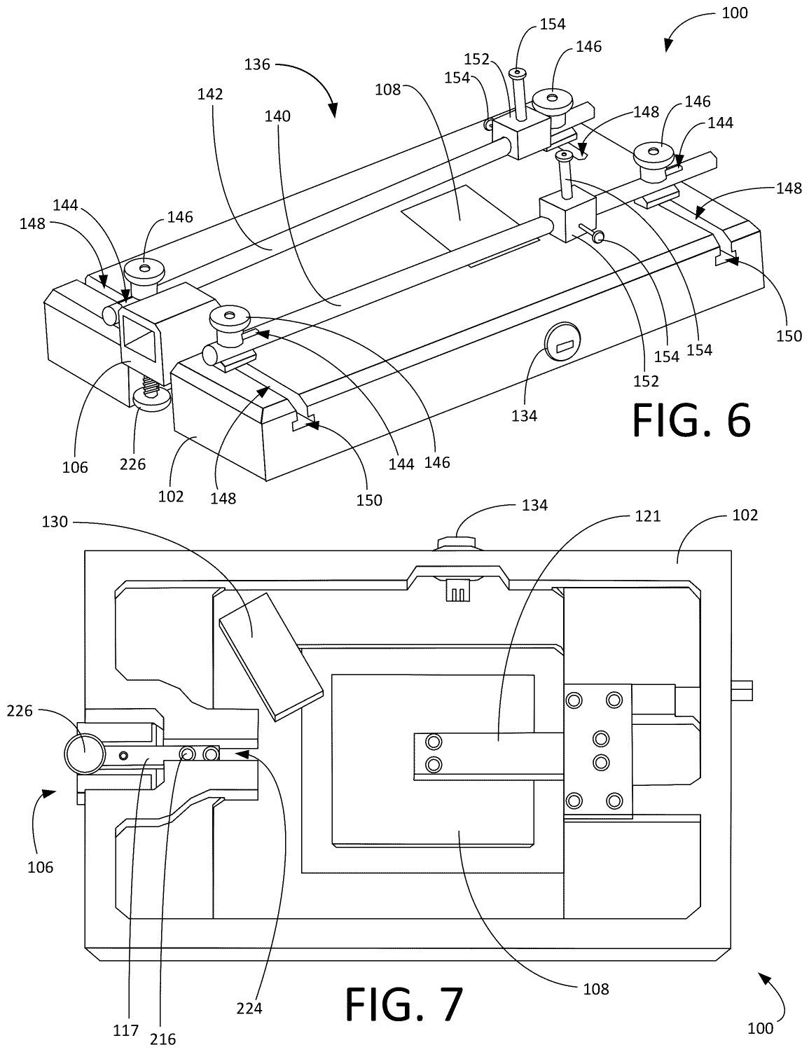

The output module 134 is configured to facilitate the exchange of information measured by the foot evaluation device 100. In one embodiment (depicted in FIG. 1), the output module 134 includes a card slot that is configured to accept a standard removable digital memory card (e.g., Secure Digital formatted cards). The memory card can be used to store and transfer measurement data from the foot evaluation device 100 to a computer. In another embodiment, as illustrated in FIGS. 6 and 7, the output module 134 includes a wired data port (e.g., USB port) to permit the direct transfer of data to and from the foot evaluation device 100 over a wired connection or using a thumb drive. In yet another embodiment, the output module 134 includes a wireless network adapter (e.g., WiFi or Bluetooth) that permits the exchange of information between the foot evaluation device 100 and a computer or network over a wireless connection. In yet other embodiments, the foot evaluation device 100 is provided with a printer configured to produce a printed report of the measurements taken and processed by the foot evaluation device 100. As used herein and in the appended claims, the term “output module” may refer to a removable memory card slot, a printer, a wired data port, and a wireless network adapter.

In some embodiments, the foot evaluation device 100 is configured as a peripheral instrument that is operable only when connected to a computer, which receives the toc-related output signals from the toe force sensor 116. In these embodiments, the foot evaluation device 100 may not include the onboard processing electronics such as the control module 112, display 124, control buttons 126, battery 128, processor 130, and memory 132.

In exemplary embodiments, the processor 130 will generate a message on the display 124 based on the heel-related and/or metatarsal-related output signals, where the user or an administering clinician is instructed to retake the toe strength measurement. In some instances, the message will indicate the likelihood of an inaccurate toe strength measurement (e.g., as a percentage). The message may also state a reason for why the measurement has been identified as inaccurate (e.g., forward movement of the foot detected at the heel).

In various embodiments, a foot placement adjustment system 136 is used to minimize movement of the user's foot during a toe strength measurement and thereby ensure that the movements detected by the toc force sensor 116 are isolated to the user's toe. The foot evaluation device 100 optionally includes a foot strap (not pictured) that can be threaded through platform apertures 138, as show in FIG. 1, and used to secure the user's foot to the platform 102. The foot strap can be threaded through different platform apertures 138 on the platform 102 depending on whether the user's left or right foot is being tested with the foot evaluation device 100. The front strap may also be adjusted to accommodate different foot sizes.

Referring now to FIG. 6, shown therein is a top perspective view of a foot evaluation device 100 having a foot placement adjustment system 136 constructed in accordance with another embodiment. The foot placement adjustment system 136 uses a first foot placement rod 140 and a second foot placement rod 142 to guide the foot and properly position the user's toc at the toe sensor assembly 106. The first foot placement rod is configured for placement against one side of the user's foot, and the second foot placement rod is configured for placement against an opposite side of the user's foot. Each of the foot placement rods 140, 142 includes a rod channel 144 at cither end, where each rod channel 144 receives a position adjustment knob 146. Each position adjustment knob 146 slides along its respective rod channel 144 to move the respective foot placement rod 140, 142 forwards or backwards along the platform 102. Each of the position adjustment knobs 146 is further disposed within a platform channel 148. The platform channels 148 are elongated recesses within the platform 102. As the position adjustment knobs 146 slide along the platform channels 148, their respective foot placement rods 140, 142 move towards and away from the center of the platform 102. By sliding the position adjustment knobs 146 along the rod channels 144 and the platform channels 148, the foot placement rods 140, 142 may be placed at different angles and orientations about the user's foot. When the foot placement rods 140, 142 are in their desired position (i.e., resting snugly against either side of the user's foot in the proper measurement position), the position adjustment knobs 146 are twisted downward to tighten, thereby securing the foot placement rods 140, 142. When a measurement is complete, the position adjustment knobs 146 may be twisted upward to loosen, thereby permitting the foot placement rods 140, 142 to be moved away from the user's foot. The terminal end 150 of each platform channel 148 is shaped and sized to allow the position adjustment knob 146 to slide out from the platform 102. Each foot placement rod 140, 142 may be removed from the platform 102 by sliding both position adjustment knobs 146 through the terminal end 150 of their respective platform channels 148.

In some embodiments, as shown in FIG. 6, the foot placement rods 140, 142 also include heel positioning blocks 152 that slide along each rod 140, 142. The heel positioning blocks 152 are placed against the back of the user's foot to prevent movement away from the toe sensor assembly 106 during a measurement. To secure each heel positioning block 152, heel knobs 154 are twisted toward the rod to increase friction and tighten the heel positioning block 152 against the foot placement rod 140, 142. To loosen the heel positioning blocks 152 when a measurement is complete, the heel knobs 154 are twisted in the opposite direction, and the heel positioning blocks 152 may be moved away from the user's foot.

Further measurements at the toe, in addition to those related to toe flexion and toc extension, are useful in diagnosing overall health of the user. To provide greater functionality, in various embodiments of the foot evaluation device 100, all or part of the toe sensor assembly 106 is detachable from the platform 102. This detachability allows different toe sensor assemblies 106 to be used with the platform 102 to perform various improved health monitoring tasks. Toc sensor assemblies 106 with toe caps 114 of various sizes or adjustable toe caps 114 are also used to accommodate and match the user's size of toe.

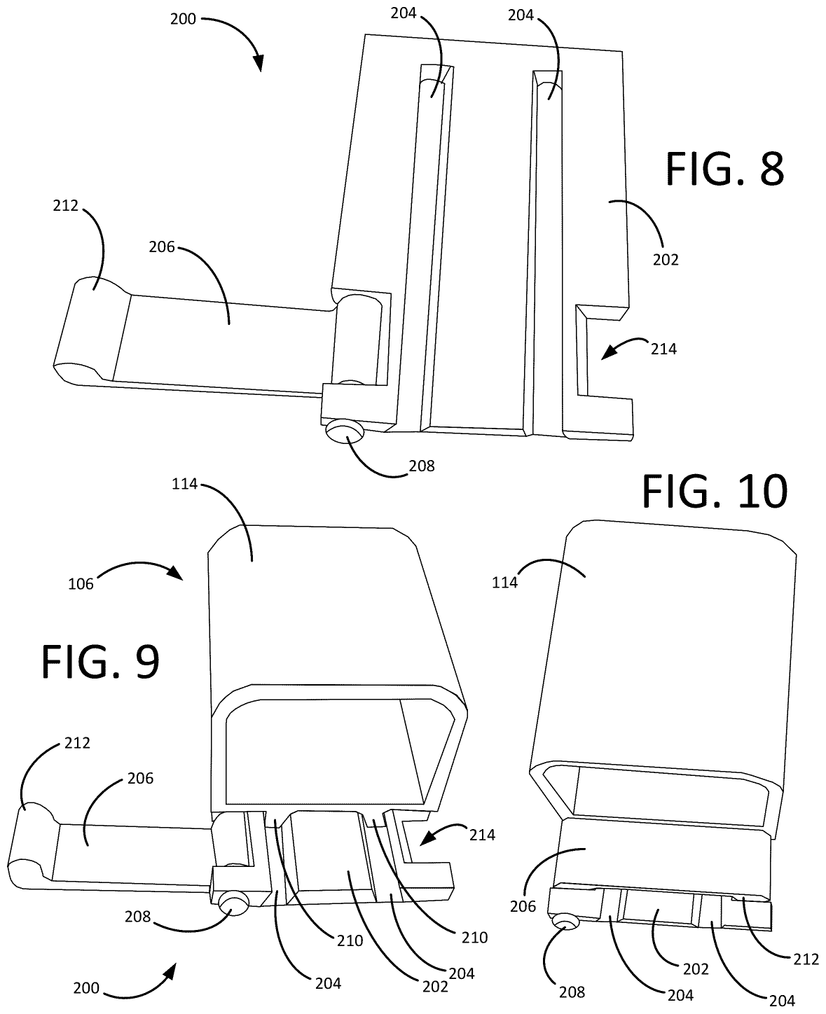

In various embodiments, one or more toe sensor assemblies 106 are removably attachable from the platform 102 and thereby used interchangeably with the foot evaluation device 100. To facilitate this exchange of different toe sensor assemblies 106, the platform 102 of the foot evaluation device 100 may include a latch assembly 200 that is configured to retain the toc sensor assembly 106. The latch assembly 200 itself may be integrated with or removably attachable from the platform 102. As depicted in FIG. 8, the latch assembly 200 includes a body 202, a plurality of rail recesses 204 disposed within the body 202, and a latching mechanism 206 that is attached to the body 202 and pivotable about a pin 208. The toe sensor assembly 106 includes a plurality of rails 210 disposed at the bottom of the toe sensor assembly 106, where each rail 210 corresponds to one of the rail recesses 204. When the latch assembly 200 is in an unlocked position, as shown in FIG. 9, the latching mechanism 206 is disposed away from the plurality of rail recesses 204 such that the rails 210 of the toe sensor assembly 106 can slide laterally along the rail recesses 204. In the locked position, the latching mechanism 206 is rotated about the pin 208 and is positioned over the plurality of rail recesses 204. As depicted in FIG. 10, when the latching mechanism 206 is placed in the locked position, the rails 210 are prevented from sliding laterally along the rail recesses 204, and the toe sensor assembly 106 is consequently secured within the latch assembly 200. To replace the toe sensor assembly 106, the latch assembly 200 is opened (i.e., the latching mechanism 206 is moved to the unlocked position by rotating about the pin 208), the existing toe sensor assembly 106 is slid from the plurality of rail recesses 204, a second toe sensor assembly 106 is slid into the plurality of rail recesses 204, and the latch assembly 200 is closed (i.e., the latching mechanism 206 is moved to the locked position).

In one embodiment, the latching mechanism 206 includes an attachment feature 212 that is received by an indentation 214 of the body 202 of the latch assembly 200. The attachment feature 212 may be shaped to snap into or otherwise engage with the indentation 214 to secure the attachment feature 212 thereagainst, such that force is required to disengage the attachment feature 212 from the indentation 214. In another embodiment, the attachment feature 212 is retained in the indentation 214 using additional fasteners (not shown) or is altogether replaced by alternative fasteners. Suitable additional or alternative fasteners include without limitation clips, flaps, clamps, pins, appendages, plugs, and hook-and-loop fasteners.

In another embodiment, the toe sensor assembly 106 is detachable from the platform 102 by virtue of one or more screws 216 disposed through corresponding toe sensor apertures 218 that are positioned at a fixed end 220 of the load cell 117 (e.g., a single-ended shear beam load cell), where the one or more screws 216 secure the toe sensor assembly 106 to the underside of the platform 102. This orientation secures the fixed end 220 of the load cell 117 against the platform 102 while a load from the user's toe is applied to a free end 222 of the load cell 117 opposite from the fixed end 220. As best illustrated in FIG. 7, in some embodiments, the underside of the platform 102 includes a channel 224 that is configured to receive the fixed end 220 of the load cell 117.

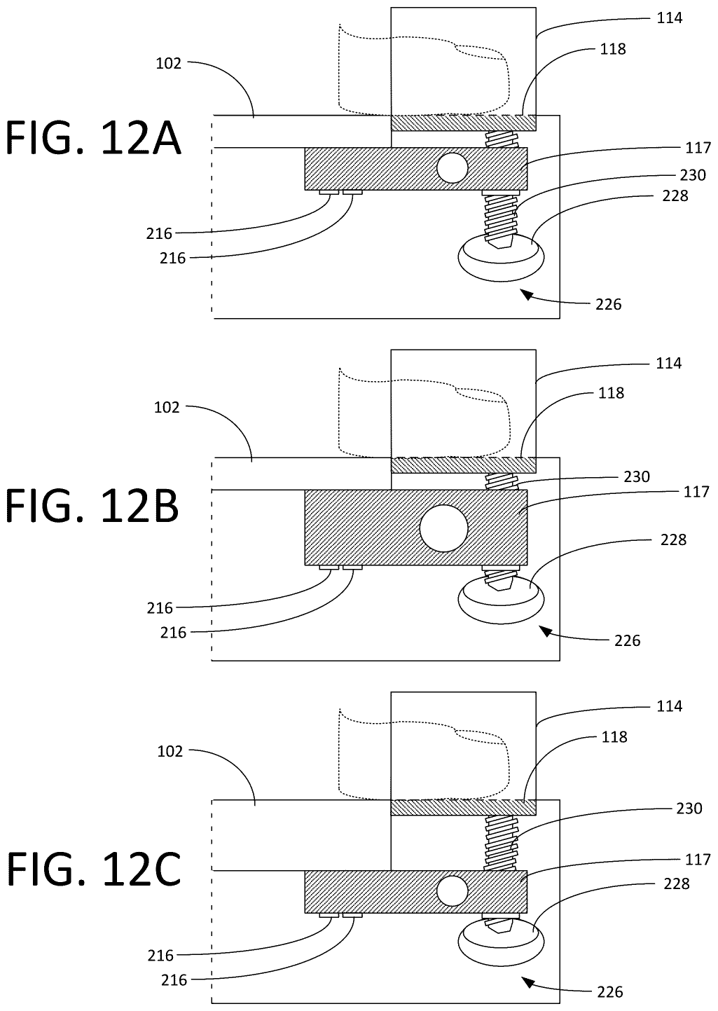

The toe sensor assembly 106 may also include a height adjustor 226 that is configured to raise or lower the toe sensor surface 118. As illustrated by FIGS. 12A through 12C, based on the widths of the platform 102 and the toe force sensor 116, the height adjustor 226 may be used to ensure that the toe sensor surface 118 is level with the surface of the platform 102. In one non-limiting embodiment, the height adjustor 226 includes a head 228 and threads 230 that are received through a hole in the free end 222 of the load cell 117. Opposite from the head 228, the height adjustor 226 interacts with the bottom of the toe force sensor 116. As the height adjustor 226 is rotated to move the threads 230 upward through the free end 222 of the load cell 117, the toe sensor surface 118 moves upward, and, conversely, when the knob is rotated to move the threads 230 downward through the free end 222, the toe sensor surface 118 moves downward. In another non-limiting embodiment, the height adjustor 226 is a plug that is manufactured from a vibration damping material, such as rubber (e.g., natural rubbers, synthetic rubbers such as ethylene propylene diene monomer (EDPM), neoprene, or butyl rubber, or a combination of the same). Suitable plug types for the height adjustor 226 include tapered plugs, round plugs, and hole plugs. The plug may be press-fit through a hole in the toe force sensor 116 and received at the bottom of the toe force sensor 116 (e.g., within a depression). In this manner, the foot evaluation device 100 may be configured to accommodate different toe sensor assemblies 106.

In one embodiment, the toe sensor assembly 106 makes a physical electronic connection with the processor 130 via one or more spring-based edge connectors 232 when the toe sensor assembly 106 is fully attached to the platform 102. The arrangement of the edge connectors 232 ensures that an electronic connection to the processor 130 is always maintained during use of the toe sensor assembly 106. In one embodiment, one or more sensors in the toe sensor assembly 106 are physically connected to the edge connectors 232 by one or more electrical wires 234, and thereby electronically connected to the processor 130. When the toe sensor assembly 106 engages with the platform 102, the electronic connection that is established with the processor allows the processor 130 to interrogate and detect the type of toe sensor assembly 106 that is in use. The detection of toe sensor assembly 106 type can be performed automatically or manually to perform testing.

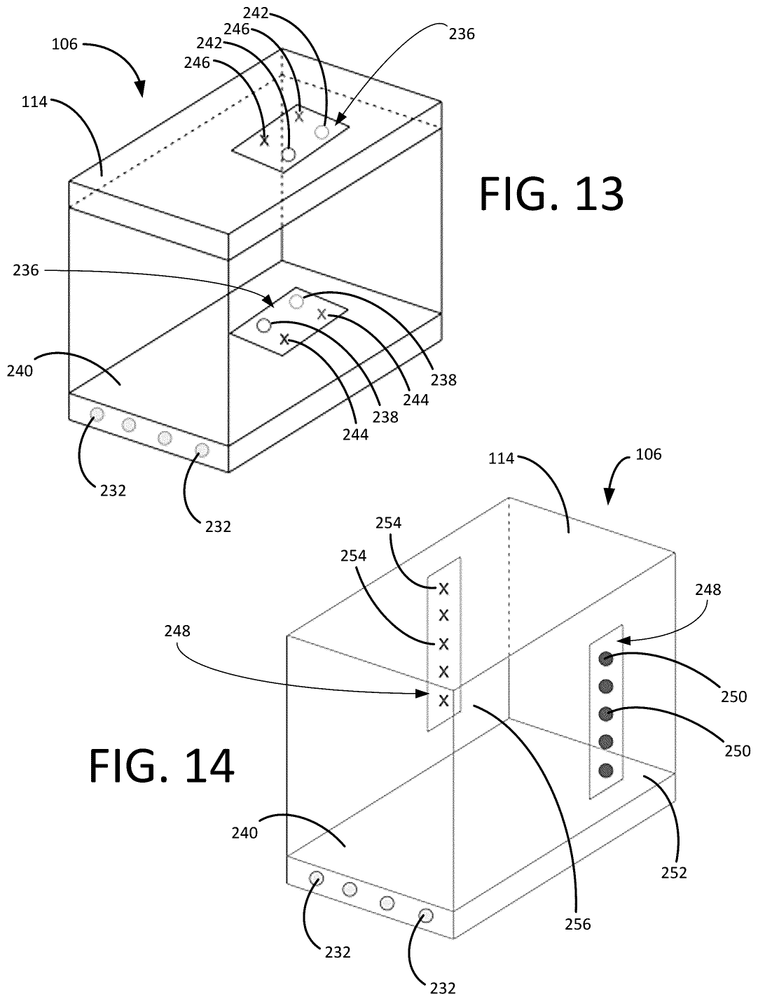

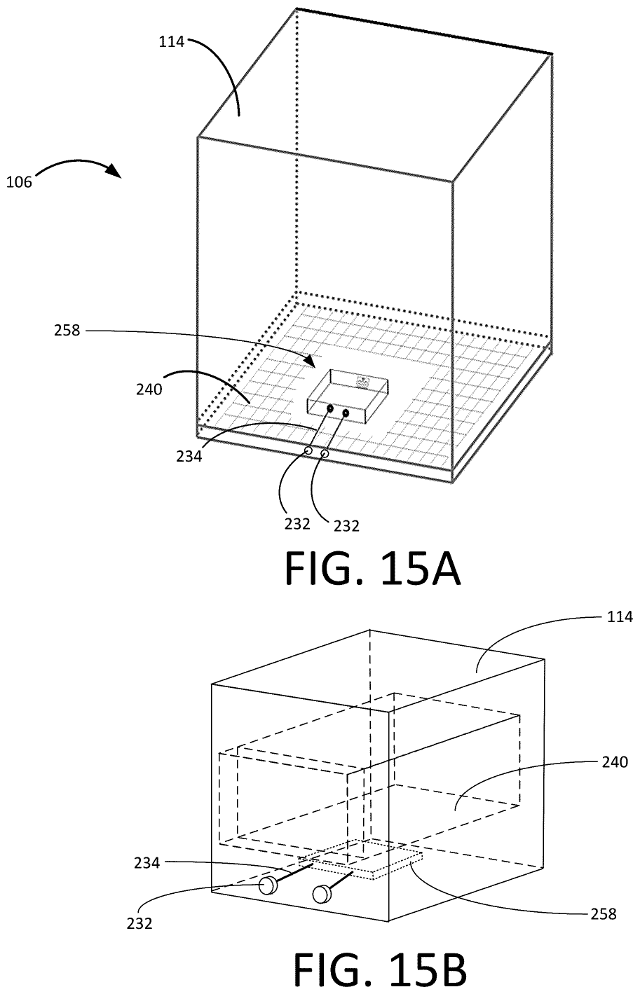

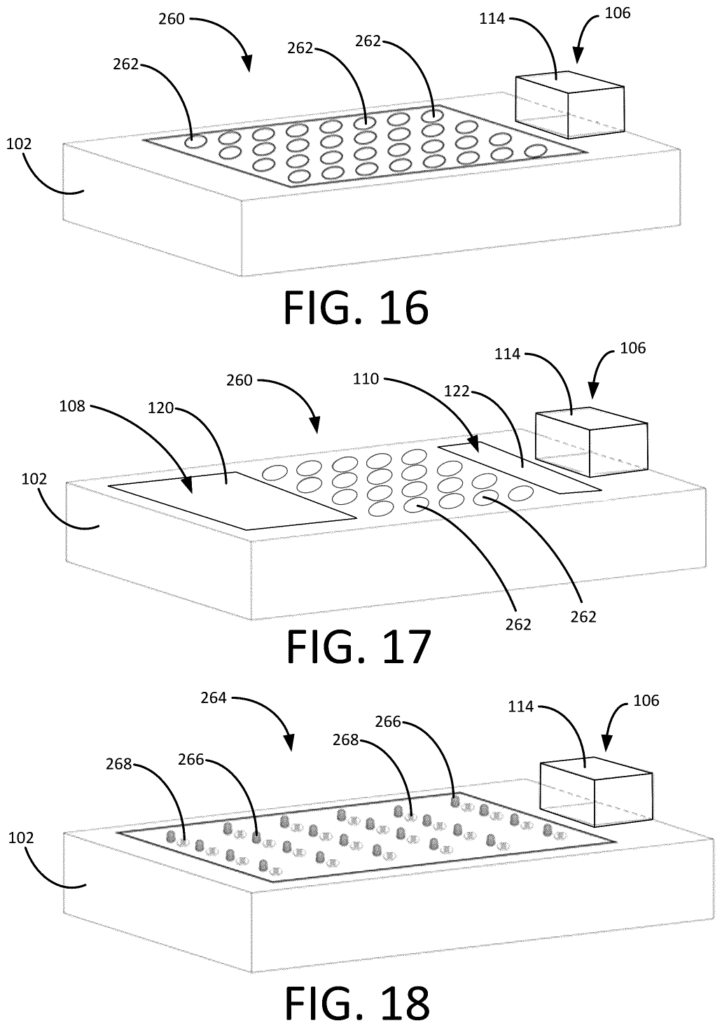

Different toe sensor assemblies 106 may be useful for diagnosing aspects of the user's overall health, in addition to toe strength and range of motion. For example, in the embodiment depicted in FIG. 13, the toe sensor assembly 106 incorporates a blood oxygen sensor 236 to measure data related to the user's circulatory system, including blood density, pulse features, and blood oxygenation levels. The blood oxygen sensor 236 includes at least one lower light emitter 238 (e.g., a red or green LED light) incorporated in a lower inner surface 240 of the toe sensor assembly 106 and at least one upper light emitter 242 (e.g., a red of green LED light) incorporated in the toe cap 114. The blood oxygen sensor 236 also includes at least one lower photodiode 244 incorporated in the toe force sensor 116 and at least one upper photodiode 246 incorporated in the toe cap 114. In use, the lower photodiode 244 and the upper photodiode 246 detect light emitted from, respectively, the upper light emitter 242 and the lower light emitter 238 when the toe is in the measurement position within the toe sensor assembly 106. The measurements of light detected by the lower and upper photodiodes 244, 246 are sent to the control module 112 for processing, interpretation, and display and can be used, for example, to determine oxygen levels in the user's toe. In another embodiment, the at least one upper photodiode 246 and the at least one upper light emitter 242 are omitted from the toe cap 114. In this embodiment, the lower light emitter(s) 238 and the lower photodiode(s) 244 are incorporated in the lower inner surface 240 of the toe sensor assembly 106, and the lower photodiode(s) 244 are calibrated to measure light that has been emitted from the lower light emitter(s) 238 and interacted subsequently with blood in the user's toe. Although two lower photodiodes 244 and lower light emitters 238 are depicted in FIG. 13, it will be appreciated the one photodiode 244 and one corresponding light emitter 238 will be suitable for some embodiments and, in other embodiments, the toe sensor assembly 106 may include more than two photodiodes 244 with more than two corresponding light emitters 238. In yet other embodiments, a plurality of photodiodes 244 is used to provide measurements at various regions of the toe with one corresponding light emitter 238.

FIG. 14 depicts another embodiment of the toe sensor assembly 106 that incorporates a toc extension assembly 248 with optics to measure the maximum extension that the user's toe can achieve. In this embodiment, the toe cap 114 is large enough to allow the user's toe to move freely forwards and upwards. It will be appreciated that, to measure maximum extension, this toc cap 114 may be taller than one that does not implement optics. At least one vertical light emitter 250 is positioned on one inner side wall 252 of the toe cap 114, while a plurality of vertical photodiodes 254 is vertically aligned on an opposite inner side wall 256. Although FIG. 14 depicts a one-to-one relationship in which each of the vertical photodiodes 254 is counter-aligned with a corresponding vertical light emitter 250 (i.e., five vertical photodiodes 254 corresponding to five vertical light emitters 250), it will be appreciated that as few as one vertical light emitters 250 is suitable to measure maximum toe extension in this toe cap 114, notwithstanding the number of vertical photodiodes 254. Resolution for the toe extension measurement depends on the spacing of the vertical photodiodes 254. For example, where the vertical photodiodes 254 are spaced 1 cm apart, the toe cap 114 provides at least 1 cm resolution. In various embodiments, the resolution of the toe cap 114 is between about 0.1 cm and about 1.5 cm. In one embodiment the resolution is about 0.2 cm. When the user's toe is in the measurement position in the toe sensor assembly 106, the at least one vertical light emitter 250 is positioned at one side of the toe, while the plurality of vertical photodiodes 254 is positioned at the opposite side of the toe. The toe cap 114 functions by measuring changes in the light detected by the vertical photodiodes 254 during a toe extension. As the toe extends vertically upward, it will block one or more of the vertical photodiodes 254, thereby reducing the amount of light measured from the at least one vertical light emitter 250. Data is collected to reflect which photodiodes 254 measured a lower level of light or no light during the toe extension. This data can be subsequently processed, interpreted, and displayed by the control module 112 to identify the maximum distance the user was able to freely extend the toc.

FIG. 15 depicts yet another embodiment of the toe sensor assembly 106 that incorporates a vibrating element 258 that may be used to detect, e.g., sensory neuropathy. Suitable vibrating elements 258 include without limitation piezoelectric actuators, linear resonant actuators, vibrating solenoids, and other motor-based vibrating devices. As depicted in FIG. 15A, the vibrating element 258 in one embodiment is positioned within the toe sensor assembly 106 so that the user's toe rests thereon. In one embodiment, the vibrating element 258 is embedded in the toe sensor assembly 106 such that the top of the vibrating element 258 is flush with the inner surface 240 of the toe sensor assembly 106. In another embodiment, the vibrating element 258 directly contacts the user's toe but is instead placed atop the inner surface 240 of the toe sensor assembly 106, creating a raised measurement surface for the user's toe. The vibrating element 258 may alternatively be placed within the toe sensor assembly 106 in a position that does not directly contact the user's toe (e.g., on or within the toe cap 114), and calculations may be made to indirectly determine the magnitude of vibrations experienced at the user's toe. As depicted in FIG. 15B, the vibrating element 258 may be positioned within a chamber underneath the position for the user's toe. Isolating the vibrating element 258 within the chamber helps to reduce the noise produced by vibrations from the vibrating element 258 during its operation. A rubber-based height adjustor 226 can also be used with toc sensor assemblies 106 that incorporate the vibrating element 258 to prevent the vibrating waves from propagating outside of the toc sensor assembly 106 to the platform 102 and thereby to other portions of the user's foot besides the toe. It will be appreciated that in other embodiments, the vibrating element 258 may be placed to the side of the platform 102 and connect to the rest of the toe sensor assembly 106 via wires or other suitable connectors to produce vibrations at the user's toe.

The vibrating element 258, when integrated within the toe sensor assembly 106, may be connected to the edge connectors 232 by the embedded electrical wires 234. This association with the edge connectors 232 may be used to power the vibrating element 258 using a vibration driver, e.g., a haptic driver (not shown). Various parameters, including but not limited to vibration patterns, frequency (rate) of vibrations, vibration amplitude (strength), and duration of the test, can be varied for the vibratory toe measurement and used to evaluated patient sensory loss. These parameters are optionally presented to the user through the display 124 or a separate visual output.

Although the foregoing embodiments describe separate toe sensor assemblies 106 used to evaluate toe flexion and toe extension (FIGS. 1 and 2), blood oxygen levels (FIG. 13), maximum toe extension (FIG. 14), sensory loss (FIG. 15), etc., it will be appreciated that the toe sensor assembly 106 can incorporate sensors to accomplish two or more of these testing protocols. For example, the toe sensor assembly 106 of one embodiment includes the vertical light emitters 250 and the vertical photodiodes 254, as well as the vibrating element 258, to accomplish two tests using one assembly 106 (e.g., a maximum toe extension test and a sensory neuropathy test). In another embodiment, the blood oxygen sensor 236 is used alongside the toe extension assembly 248. In various embodiments, the toe force sensor 116 for toe flexion and toe extension measurements (see, e.g., FIGS. 1 and 2) accompanies one or more of the sensor arrangements from FIGS. 9 through 11; in such embodiments, the toe sensor surface 118 substitutes the inner surface 240 of the toe sensor assembly 106. In one embodiment, all of the aforementioned toe-related sensors are incorporated into one toe sensor assembly 106.

Turning to additional features of the foot evaluation device 100, the platform 102 can also be modified to evaluate additional aspects of the user's overall health. For example, for certain users, such as patients undergoing chemotherapy, it is useful to understand whether orthotics or other treatment are necessary to address foot deformation. Pes planus (commonly known as “fallen arches,” “flat foot” or “flat feet”) is a condition in which the arch on the inside of one's foot flattens under downward pressure. Turning to FIG. 16, shown therein is an embodiment of the foot evaluation device 100 in which a pressure sensor array 260 is incorporated into the platform 102 to measure and produce signals indicative of flat foot, progression toward flat foot, or both. In another embodiment, the pressure sensor array 260 is a separate attachment that is placed on top of the platform 102 and is connected to the platform 102 by a wired or wireless connection. The pressure sensor array 260 includes a plurality of pressure sensors 262, each of which is substantially flush with the surface of the platform 102 when embedded therein. In exemplary embodiments, the pressure sensors 262 are Tekscan™ pressure sensors or pressure mapping sensors. Features of the user's foot can be determined based on differences in the force applied to the pressure sensors 262 when the pressure sensors 262 contact the user's foot at different points on the platform 102. For example, elevated pressure levels between the user's heel and metatarsal may indicate downward progression of the foot's arch. In various embodiments, data from the pressure sensor array 260 is sent to the processor 130 as pressure array output signals. This data can be used by the control module 112 to capture a template of the user's foot, a pressure map, or both. The pressure sensor array 260 is optionally calibrated before and/or between measurements to improve accuracy.

As shown in FIG. 16, the pressure sensor array 260 can be incorporated into the platform 102 without the heel force sensor 108 and/or the metatarsal force sensor 110. Without the heel force sensor 108 and metatarsal force sensor 110, the pressure sensor array 260 is positioned on a solid surface and is configured to measure and record data related to flat foot progression.

FIG. 17 depicts an exemplary embodiment of the pressure sensor array 260 incorporated alongside the heel force sensor 108 and the metatarsal force sensor 110. When the pressure sensor array 260 is incorporated with these force sensors 108 and 110, the pressure sensors 262 provide less accurate pressure measurements. Nevertheless, the pressure sensors 262 still measure and report data that can be used to detect flat foot, progress toward flat foot, or both.

The pressure sensor array 260 may alternatively be a removable attachment that is placed on top of the platform 102. If the pressure sensor array 260 is placed atop the platform 102 and covers the heel force sensor 108 and the metatarsal force sensor 110, whether measurements are still obtained from the sensors 108, 110 depends on the material used for the plate of the pressure sensor array 260. A metal plate will prevent heel and metatarsal force measurements, while a plate made from a flexible material (e.g., a plastic in which the pressure array is embedded) will permit heel and metatarsal force measurements.

In other exemplary embodiments, optics within the platform 102 of the foot evaluation device 100 can be used to detect progression toward flat foot. Turning to FIG. 18, an embodiment of the foot evaluation device 100 is shown in which an optical sensor array 264 is incorporated in the platform 102 and configured to monitor flat foot progression. The optical sensor array 264 includes a plurality of optics devices. In one embodiment, the optical sensor array 264 includes a plurality of light emitters 266, such as LED lights, and a plurality of photodiodes 268. Based on the shape and placement of the user's foot, different photodiodes 268 will be able to detect stronger light signals than others when light from the light emitters 266 is reflected by the user's foot. Areas where the surface of the foot is raised further above the platform 102 will allow for greater light transmission between the light emitters 266 and the surrounding photodiodes 268. The optical sensor array 264 thereby measures and produces one or more signals indicative of flat foot, and these signals are conveyed to the control module 112 for processing, interpretation, and display. This data can be used by the control module 112 to capture a template of the user's foot. Where extensive data collection is possible, the optical sensor array 264 can also be used to estimate pressures applied by the user's foot to the optical sensor array 264. For example, neural networks and machine learning processes can be used to correlate pressure levels applied by the foot with sensor data received from the optical sensor array 264. In one embodiment, the data acquired from the optical sensor array 264 can be further used to measure distribution of oxygen within the foot. Data from the optical sensor array 264 may also be used to determine thickness of the skin. Where the skin is more opaque, lower levels of light will pass through and, therefore, a larger measurement will be obtained for light that is reflected from the foot. Such skin thickness measurements may be used as an indicator for conditions such as hyperkeratosis, skin cancer, or bacterial or viral infections on the skin.

In various embodiments, the foot evaluation device 100 includes a pedal attachment 300, which allows for additional measurements related to the user's foot. Foot push measurements, for example, can be used to characterize the strength of the user's foot muscles. In one embodiment, the foot evaluation device 100 is used to determine the maximum strength that the user's foot can exert. In other various embodiments, a curve is generated based on the pressure exerted by the user's foot over time, and aspects of the curve (e.g., rise to peak, shape of the curve) are used as indicators for conditions such as amyotrophic lateral sclerosis (“ALS”) or as indicators for the user's response to treatments such as those for neuropathy or cancer.

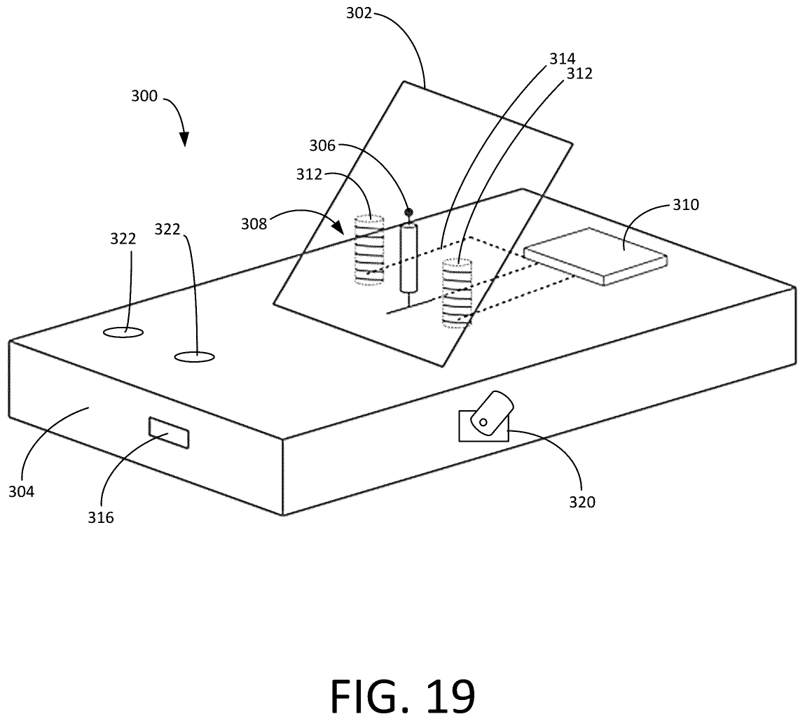

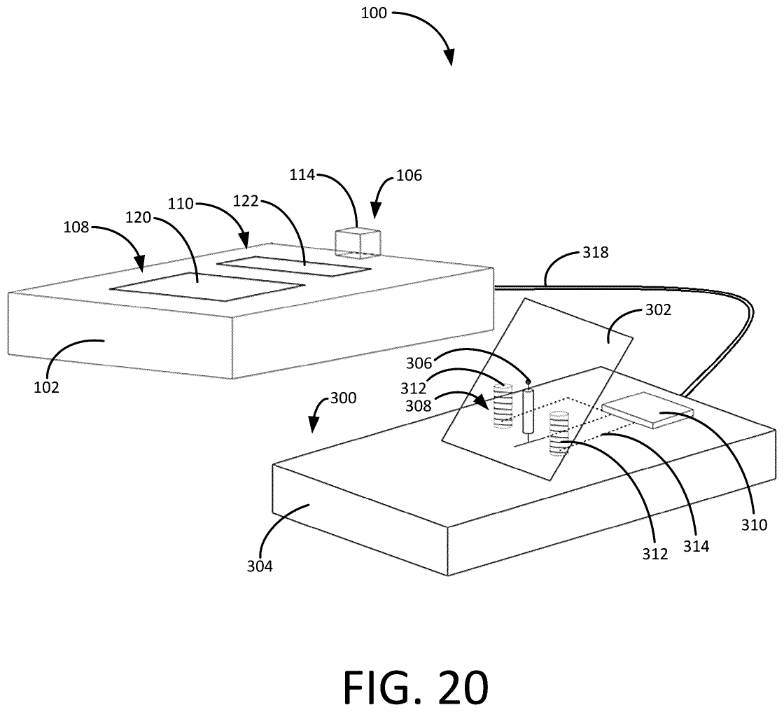

The pedal attachment 300 includes a foot pedal 302 connected to a base 304, a potentiometer 306 connected to the foot pedal 302, a linear actuator 308 connected to the foot pedal 302, and a local processor 310 in communication with the potentiometer 306 and the linear actuator 308. In the exemplary embodiments depicted in FIGS. 19 and 20, the linear actuator 308 is an electrical linear actuator that is positioned underneath the foot pedal 302. The electrical linear actuator 308 includes one or more controllable electrical coils 312 used to create variable resistance. In this way, the coils 312 can be adjusted by the local processor 310 to increase or decrease the resistance based on the current applied. In one embodiment, the potentiometer 306 and the linear actuator 308 are connected to the local processor 310 by an I2C bus 314. The pedal attachment 300 interfaces with the control module 112 of the foot evaluation device 100 by a data port 316 (e.g., a USB port), a wired connection 318, or through wireless connection (e.g., Bluetooth wireless).

To obtain a foot push measurement, the user's foot is positioned on the foot pedal 302 and exerts a downward force to pivot the foot pedal 302 toward the platform 102 against the resistance applied by the coils 312. During the foot push measurement, the linear actuator 308 provides resistance against the user's foot and the pedal 302, where the level of resistance is controlled by the local processor 310. The level of resistance can be varied depending on the individual user and test protocol. In one embodiment, the level of force resistance is directly selected through the control module 112. In another embodiment, the proper resistance is determined through an automated procedure whereby the processor 130 of the foot evaluation device 100 executes an algorithm and uses a feedback control circuit to calculate an appropriate level of resistance based on inputs, such as the user's age. In one embodiment, the amount of resistive force is modulated to find a point of equilibrium between the force applied by the user's foot to the foot pedal 302 and the level of resistance. If the user is pushing at maximum strength at the point of equilibrium, the level of resistance will directly correspond to the maximum strength of the user's foot muscles. The potentiometer 306 is configured to measure the downward applied force on the foot pedal 302 and produce foot push signals representative of the magnitude of the applied force.

In one embodiment, the pedal attachment 300 relays the foot push signals to the control module 112 of the foot evaluation device 100 for processing, interpretation, and display. In another embodiment, the local processor 310 of the pedal attachment 300 is configured to interpret the foot push signals. The foot push signals can be presented to the user as an actual measurement of force (absolute) or as a scaled force measurement (e.g., out of a scale of 1 to 10).

Evaluation of sensory and motor nervous systems for the foot muscles can also be performed by applying external stimulation to the user's foot. One embodiment of the pedal attachment 300 includes a vibratory motor 320 attached to the foot pedal 302. When energized, the motor 320 induces vibratory motion to stimulate the foot muscles' sensory and motor nervous systems. In another embodiment, the foot pedal 302 includes a plurality of terminals 322 positioned to contact various parts of the foot. These terminals 322 apply an AC current to stimulate sensory and motor response of the foot nervous system. Signals representative of the foot muscles' sensory and motor nervous systems are sent to the processor 130 of the control module 112 or to the local processor 310 for interpretation.

In one embodiment, a method of operation of the present disclosure begins by placing the foot evaluation device 100 on the floor or other stable surface. The user's foot is positioned on the platform 102 such that the user's foot is in contact at the heel with the heel force sensor 108 and the first toe is located under the toc cap 114. In this embodiment, the user places his own foot on the platform 102 and performs a measurement protocol without the assistance of a trained clinician. It will be appreciated, however, that in other embodiments the user is assisted by a clinician, as necessary. The toe cap 114 can be adjusted or exchanged to ensure a proper fit with the user's first toe. Once the user's toe is secured within the toe cap 114, the toe force sensor 116 is tared and the user executes the measurement protocol. In some embodiments, software is developed to instruct the user of the steps required to perform the measurement protocol. The software can also inform the user of improper measurement and recommend retaking the measurement. The software can also auto-invalidate the measurement if certain parameters of the measurement protocol are not met. Depending on the measurement protocol, the user's toe strength is evaluated by asking the user to perform toe flexion and toe extension maneuvers while the foot evaluation device 100 records real time measurements that are representative of the forces applied to the toe force sensor 116. Heel-related measurements are simultaneously recorded for forces applied to the heel force sensor 108 during the measurement protocol. At the conclusion of the examination protocol, the user's foot is released from the platform 102. The measurement data recorded by the foot evaluation device 100 can then be shared from the output module 134 for further analysis and diagnostics.

In another embodiment, metatarsal-related measurements are recorded during the measurement protocol in addition to or in place of the heel-related measurements, and these measurements represent the forces applied to a metatarsal force sensor 110.

In another embodiment, the user is instructed to detach the toe sensor assembly 106 after performing toe flexion and toe extension maneuvers and to insert a second toe sensor assembly 106 into the foot evaluation device 100. This process may involve the user unlocking and locking a latch assembly 200. The second toe sensor assembly 106 includes at least one of the blood oxygen sensor 236, the toe extension assembly 248, and the vibrating element 258. The user is then instructed to perform one or more additional measurements for the measurement protocol using the second toe sensor assembly 106.

In another embodiment, the user's foot is positioned on the platform 102, and additional metrics of the user's health are obtained for the measurement protocol, either before or after toe flexion and toe extension maneuvers are performed, using the pressure sensor array 260, the optical sensor array 264, or both.

In another embodiment, the measurement protocol requires foot push maneuvers to be conducted on a pedal attachment 300 to the foot evaluation device 100, either before or after toc flexion and toe extension maneuvers are performed. The user's foot is positioned on a foot pedal 302 of the pedal attachment 300, and the user is instructed to push downward with the user's foot to pivot the foot pedal 302 toward the platform 102. Depending on the measurement protocol, the user may be instructed to push downward at different intervals, for different durations, and against different levels of force resistance.

The foot evaluation device 100 is well suited to record the user's ability to exert force over an extended period (for the functionally relevant estimation of muscle or nerve fatigue). A typical strength profile recorded by the foot evaluation device 100 is illustrated, in one example, as a graph plotting the force exerted by the user's toe on the toe force sensor 116 (in Newtons) over time (in seconds). The foot evaluation device 100 can be configured to analyze the data recorded during the measurement session and extract certain metrics or health indices based on the data recorded by the foot evaluation device 100. These metrics include, but are not limited to, peak strength, time to peak, rate of increase up to peak strength, and the rate of decrease after peak strength. The foot evaluation device 100 can also be configured to determine the degree of unsteadiness in strength profile. Various signal processing techniques and statistical methods may be used in compiling these metrics and indices.

The foot evaluation device 100 can also be configured to aggregate data from multiple measurement session profiles for easy reference and comparison. This data is stored in one embodiment in a cloud-based server. As noted above, the software to perform all these measurement, analytic and display functions may reside on the foot evaluation device 100 itself or on an external device such as a remote microcontroller board, a smart phone, a smart watch, a tablet, a cell phone, a single board computer, a laptop computer, a desktop computer, or a cloud server computer. The software may compute the metrics automatically or allow a user to generate metrics based on customized requirements.

Thus, the embodiments of the present disclosure are well adapted to carry out the objects and attain the ends and advantages mentioned above as well as those inherent therein. While the inventive device and system have been described and illustrated herein by reference to particular non-limiting embodiments in relation to the drawings attached thereto, various changes and further modifications, apart from those shown or suggested herein, may be made therein by those of ordinary skill in the art, without departing from the spirit of the inventive concepts.

What is claimed is:

1. A foot evaluation device comprising:
a platform configured to accommodate a user's foot; and
a toe sensor assembly that is removably attachable to the platform, wherein the toe sensor assembly comprises:
a toe force sensor configured to measure forces applied to the toe force sensor by a toe of the user's foot, and
a toe cap connected to the toe force sensor.

2. The foot evaluation device of claim 1, wherein the toe sensor assembly is configured to measure and produce toe-related output signals representative of the magnitude and direction of the forces applied to the toe force sensor by toe flexion and toe extension.

3. The foot evaluation device of claim 2, further comprising a heel force sensor comprising a heel sensor surface, wherein the heel force sensor is configured to measure and produce heel-related output signals representative of forces applied by the user's foot to the heel sensor surface during toe flexion and toe extension.

4. The foot evaluation device of claim 3, further comprising a control module that is configured to estimate the accuracy of measurements taken by the toe sensor assembly using the heel-related output signals.

5. The foot evaluation device of claim 3 further comprising a metatarsal force sensor comprising a metatarsal sensor surface, wherein the metatarsal force sensor is configured to measure and produce metatarsal-related output signals representative of forces applied by the user's foot to the metatarsal sensor surface during toe flexion and toe extension.

6. The foot evaluation device of claim 5, further comprising a control module that is configured to estimate the accuracy of measurements taken by the toe sensor assembly using the heel-related output signals and the metatarsal-related output signals.

7. The foot evaluation device of claim 1, further comprising a foot placement adjustment system, wherein the foot placement adjustment system comprises:
a first foot placement rod configured for placement against one side of the user's foot; and
a second foot placement rod configured for placement against an opposite side of the user's foot.

8. The foot evaluation device of claim 1, further comprising a latch assembly that is configured to removably attach the toe sensor assembly to the platform, wherein the toe sensor assembly further comprises a plurality of rails, the latch assembly comprising:
a body;
a plurality of rail recesses disposed within the body, wherein each of the plurality of rail recesses corresponds to one of the plurality of rails for the toe sensor assembly; and
a latching mechanism attached to the body, wherein the latching mechanism prevents the plurality of rails from sliding along the plurality of rail recesses in a locked position.

9. The foot evaluation device of claim 1, wherein the toe sensor assembly further comprises a height adjustor that is configured to raise or lower a toe sensor surface of the toe force sensor.

10. The foot evaluation device of claim 1, further comprising a second toe sensor assembly that is removably attachable to the platform.

11. The foot evaluation device of claim 1, further comprising a blood oxygen sensor, wherein the blood oxygen sensor comprises:
at least one light emitter, and
at least one photodiode, wherein the at least one photodiode detects light emitted from the at least one light emitter.

12. The foot evaluation device of claim 1, wherein the toe sensor assembly further comprises a toe extension assembly, wherein the toe extension assembly comprises:
at least one vertical light emitter on one inner side wall of the toe cap; and
a plurality of vertical photodiodes aligned vertically on an opposite inner side wall of the toe cap.

13. The foot evaluation device of claim 1, further comprising a vibrating element.

14. The foot evaluation device of claim 1, further comprising a pressure sensor array, wherein the pressure sensor array comprises a plurality of pressure sensors that contact the user's foot at different points on the platform.

15. The foot evaluation device of claim 1, further comprising an optical sensor array that comprises a plurality of light emitters and a plurality of photodiodes.

16. The foot evaluation device of claim 1 further comprising a pedal attachment, wherein the pedal attachment comprises:
a foot pedal;
a potentiometer connected to the foot pedal, where the potentiometer measures an applied force by the user's foot to the foot pedal;
a linear actuator connected to the foot pedal; and
a processor in communication with the potentiometer and the linear actuator.

17. A method for evaluating a user's foot with a foot evaluation device, the method comprising the steps of:
attaching a toe sensor assembly to a platform of the foot evaluation device;
positioning a toe of the user's foot in contact with a toe force sensor of the toe sensor assembly;
instructing the user to execute a measurement protocol that includes toe flexion exercises, toe extension exercises, or a combination of toe flexion and toe extension exercises;
recording toe-related measurements resulting from the application of force by the user's toe on the toe force sensor during the measurement protocol; and
detaching the toe sensor assembly from the platform.

18. The method of claim 17, further comprising the steps of:
recording heel-related measurements resulting from the application of force by the user's foot on a heel force sensor during the measurement protocol, and
analyzing the heel-related measurements over time to estimate the accuracy of the toe-related measurements.

19. The method of claim 17, further comprising the steps of:
attaching a second toe sensor assembly to the foot evaluation device, wherein the second toe sensor assembly includes at least one of a blood oxygen sensor, a wall sensor assembly, and a vibrating element; and
obtaining a metric of the user's health from the second toe sensor assembly.

20. The method of claim 17, further comprising the steps of:
obtaining a foot push measurement from a pedal attachment of the foot evaluation device, and
using the foot push measurement to characterize strength of the user's foot muscles.

21. A foot evaluation device comprising:
a platform configured to accommodate a user's foot;
a foot placement adjustment system configured to minimize movement of the user's foot on the platform;
at least one toe sensor assembly that is removably attachable to the platform;
a heel force sensor positioned along the platform to contact the heel of the user's foot when the toe is within the toe sensor assembly, wherein the heel force sensor is configured to measure and produce heel-related output signals representative of forces applied by the user's foot to the heel force sensor during toe flexion and toe extension;
a metatarsal force sensor positioned along the platform to contact the metatarsal region of the user's foot when the toe is within the toe sensor assembly, wherein the metatarsal force sensor is configured to measure and produce metatarsal-related output signals representative of forces applied by the user's foot to the metatarsal force sensor during toe flexion and toe extension;
a pressure sensor array, wherein the pressure sensor array comprises a plurality of pressure sensors that contact the user's foot at different points on the platform;
an optical sensor array that comprises a plurality of light emitters and a plurality of photodiodes that contact the user's foot on the platform;
a control module that is configured to estimate the accuracy of measurements taken by the toe sensor assembly using the heel-related output signals and the metatarsal-related output signals; and
a pedal attachment that interfaces with the control module, wherein the pedal attachment comprises:
a foot pedal;
a potentiometer connected to the foot pedal, where the potentiometer measures an applied force by the user's foot to the foot pedal;
a linear actuator connected to the foot pedal; and
a processor in communication with the potentiometer and the linear actuator.

22. The foot evaluation device of claim 21, wherein the at least one toe sensor assembly is selected from the group consisting of:
(i) a toe sensor assembly comprising:
a toe force sensor configured to measure forces applied by a toe of the user's foot to the toe force sensor, and
a height adjustor that is configured to raise or lower the toe force sensor;
(ii) a toe sensor assembly comprising a blood oxygen sensor, wherein the blood oxygen sensor comprises a light emitter and a corresponding photodiode;
(iii) a toe sensor assembly comprising:
a toe cap, and
a toe extension assembly comprising a vertical light emitter positioned on an inner side wall of the toe cap and a vertical photodiode that is aligned with the vertical light emitter on an opposite inner side wall of the toe cap; and
(iv) a toe sensor assembly comprising a vibrating element.
